(19) United States
(12) Patent Application Publication
(10) Pub. No.: US 2005/0182021 A1
(43) Pub. Date: Aug. 18, 2005

(54) Polysaccharide alcohol antiseptic gel

(76) Inventors: Everett J. Nichols, Edmonds, WA (US); James R. Scott, Bellevue, WA (US)

Correspondence Address: CHRISTENSEN, O'CONNOR, JOHNSON, KINDNESS, PLLC, 1420 FIFTH AVENUE, SUITE 2800, SEATTLE, WA, 98101-2347, US

(21) Appl. No.: 11/036,594

(22) Filed: Jan. 13, 2005

Related U.S. Application Data
(60) Provisional application No. 60/536,096, filed on Jan. 13, 2004.

(51) Int. Cl. A61K 31/715; A61K 31/555; A61K 31/14

(52) U.S. Cl. 514/54; 514/345; 514/642; 514/55; 514/184

(57) ABSTRACT

An alcohol-based composition for sanitizing surfaces has persistent sanitizing properties after evaporation of the alcohol. The composition includes a cationic polysaccharide, an organic acid, alcohol, and water. The cationic polysaccharide can be chitosan, cationic guar or cationic starch. The cationic polysaccharide can be complexed with a transition metal, such as zinc or copper, or with a chelating agent, such as pyrithione.

### CROSS-REFERENCE TO RELATED APPLICATION

This application claims the benefit of U.S. Provisional Patent Application No. 60/536,096, filed on Jan. 13, 2004, incorporated herein expressly by reference.

### FIELD OF THE INVENTION

This invention relates to a composition incorporating a soluble cationic polysaccharide, such as chitosan, in alcohol, to form a gel that can be used for sanitizing skin and inanimate surfaces. Other cationic polysaccharides include cationic guar and cationic starch. The alcohol-based composition may include a combination of one or more of any cationic polysaccharides.

### BACKGROUND OF THE INVENTION

Hospital-acquired or nosocomial infections are a major concern in healthcare facilities. Hand hygiene is a significant factor in helping to prevent the spread of infection. Although hand washing is effective, the compliance of healthcare workers with hand washing guidelines involving soap and water is less than ideal. The lack of compliance with hand washing guidelines has been addressed and much progress has been made through the use of alcohol-based instant hand sanitizers. Studies have shown that hand rubbing with an alcohol based-rinse or gel is more effective in reducing hand contamination compared to hand washing with antiseptic soap. Evidence exists demonstrating that alcohol-based gels are less efficient than alcohol-based rinses in bacterial reduction capability. The lower antibacterial efficiency of gels is attributed to the presence of emollients, such as glycerol. Emollients are added to alcohol-based hand gels in order to eliminate the drying effects of the alcohol on skin.

The Food and Drug Administration has defined in The Tentative Final Monograph for Health-Care Antiseptic Drug Products; Proposed Rule C.F.R. Parts 333 and 369, titled Topical Antimicrobial Drug Products for Over-the-Counter Human Use, that the “antimicrobial agent is broad spectrum and if possible, that the antimicrobial effect is persistent.” As stated on pages 13-14 of this Monograph, the “agency agreed with the Panel that persistence, defined as prolonged activity, is a valuable attribute that assures antimicrobial activity during the interval between washings and is important to a safe and effective health-care personnel handwash.” (43 Fed. Reg. 1215.) The Panel explained that a property such as persistence, which acts to prevent the growth or establishment of transient microorganisms as part of the normal baseline or resident flora, would be an added benefit. (39 Fed. Reg. 33103 at 33115.) Although the Panel did not propose persistence as a mandatory requirement for a health-care personnel handwash, the agency is retaining the words “if possible, persistent” in the definition in this tentative final monograph because this is a desirable trait for these products.

Since alcohol alone does not provide persistence, the inclusion of an antimicrobial, such as triclosan, has been studied. A test product consisting of 0.5% triclosan in 60% n-propyl alcohol was compared to 60% n-propyl alcohol alone (control) for reduction of normal baseline flora and persistence of the reduction for a time period of 3 hours on the hands of 15 test subjects. The results of this test showed that both test product and control immediately reduced the baseline number of bacteria by approximately 99.5%. After 3 hours, the test product containing 0.5% triclosan allowed only about a one-fold increase in bacterial count while the alcohol-only control allowed an approximately twelve-fold increase.

A number of patents have been issued that describe a method for making water soluble chitosan salts. See, for example, U.S. Pat. Nos. 5,061,792; 4,574,150; and 4,929,722; and United Kingdom application GB2107340A, all expressly incorporated herein by reference. U.S. Pat. No. 5,061,792, issued to Albisetti, describes the heterogeneous reaction between particulate chitosan and an acid, suspended in an alcohol water mixture. After the reaction is completed, the water and alcohol is removed to recover the chitosan salt. However, adding chitosan to the water/alcohol mixture before the addition of the acid, prevents chitosan from dissolving in water due to the presence of the alcohol. Patent application GB 2,107,340A describes an aqueous alcohol solution of chitosan with a surfactant of a specific chemical formula to assist the dissolution of chitosan. Furthermore, the alcohol level described in the GB 2,107,340A application is not sufficient to render the solution a sanitizer.

Accordingly, there is a need to develop sanitizers having persistent antimicrobial activity. The present invention provides a sanitizing composition having persistent antimicrobial effects, and has further related advantages.

### SUMMARY OF THE INVENTION

The present invention is related to an alcohol-based sanitizing composition containing chitosan alone or in combination with other cationic polysaccharides. The composition also includes alcohol and an organic acid. The composition is at least 60% alcohol by volume. The composition may optionally include transition metals, chelating agents, and surfactants. The composition of the invention can be made as a gel for use as a hand lotion. The sanitizing composition according to the invention provides “persistence,” as defined supra. One embodiment of the composition provided no detectable increase in bacterial population, measured 4 hours after the composition was intentionally challenged with bacteria, and the composition had been allowed to dry prior to being challenged. However, the composition of the invention may exhibit persistence in excess of 4 hours. Zinc ions and pyrithione can further enhance the persistence of the antimicrobial activity of the sanitizing composition.

The alcohol-based sanitizing composition according to the invention can be used by health care personnel, food service establishment personnel, pharmaceutical manufacturing personnel, food manufacturing personnel, animal health care personnel, and the general public for sanitizing skin surfaces, including the hands. The sanitizing composition according to the invention may also be used as a surgical scrub to cleanse skin surfaces prior to surgical procedures. It is also envisioned that the sanitizing composition can be applied to solid surfaces, such as doorknobs, pens, countertops, and other solid surfaces subjected to repeated contact by the public. The sanitizing composition is thought to enhance the killing of bacteria and other microorganisms, such as fungi, protozoa (protozoa oocysts), and viruses.

One embodiment of the sanitizing composition according to the present invention is made from one or more cationic polysaccharides, such as chitosan, cationic guar, cationic starch, or a combination. The composition may also include an alcohol, such as ethanol or isopropanol, or a combination; an organic acid, such as lactic acid, pyrrolidonecarboxylic acid, acetic acid, glutamic acid, malic acid, succinic acid, fumaric acid or glucuronic acid, the salts thereof, or any combination; and water. In some embodiments, the sanitizing composition may include a transition metal, such as zinc, copper, or a combination, wherein the transition metal is complexed with the cationic polysaccharide. In other embodiments, the sanitizing composition may include a chelating agent, such as pyrithione. In still other embodiments the sanitizing composition can include a surfactant to disrupt the membranes of microorganisms.

The composition according to the invention can be applied to a surface as a solution or gel. After the alcohol and water dissipates from the surface to which the composition has been applied, the chitosan polymer or chitosan polymer in combination with any other cationic polymers, such as cationic guar and/or cationic starch, or in combination with zinc and pyrithione, remains on the surface providing a cationic environment that inhibits antimicrobial activity and prevents microbial growth and is not conducive to microbial growth. The present invention provides an alcohol-based sanitizing composition that reduces microorganisms on contact and also exhibits persistence of the antimicrobial activity after the alcohol has evaporated. Chitosan provides natural antibacterial activity and inhibits the growth of a variety of bacteria including Corynebacterium, which is typically present on skin and in sweat.

A method of making the sanitizing composition includes adding a polysaccharide to water; adding an organic acid to cause the polysaccharide to dissolve in the water; and adding alcohol to the water to form an alcohol-based composition that has the ability to kill microorganisms on contact and further prevent an increase of microorganisms after the alcohol has evaporated from the surface to which the sanitizing composition has been applied. In contrast to the prior art, the method according to the present invention, first dissolves the chitosan in water due to the addition of an acid, and is followed by the slow addition of alcohol to be able to raise the concentration of the alcohol to at least 60% by volume. The composition according to the invention can be applied to surfaces, leaving behind a chitosan salt film after evaporation of the water and alcohol to render a surface biocidal.

Chitosan advantageously counteracts the drying effects of alcohol. Chitosan is bioadhesive to skin and hair and provides a clear protective coating or film that minimizes moisture loss from skin. Since alcohol has a drying effect on skin, the inclusion of chitosan as a skin moisturizer or humectant provides benefits not present in conventional sanitizers with emollients. Chitosan also increases skin flexibility and elasticity and reduces skin irritation caused by preservatives or fragrances that might be included in conventional hand gels.

### DETAILED DESCRIPTION OF THE PREFERRED EMBODIMENT

The present invention is related to a composition that can be used as a sanitizing gel for solid surfaces, and particularly for use as a hand sanitizer. The composition includes a cationic polysaccharide, an alcohol, an organic acid, and water. The composition provides persistent antibacterial effects after the composition is dry and the alcohol and water have substantially or completely evaporated from the surface to which the composition has been applied. The composition according to the invention may optionally include a transition metal, a chelating agent, and a surfactant. The composition according to the invention may further include one or more antimicrobial compounds in combination with the cationic polysaccharide. Representative of the compounds that can be combined, include, but are not limited to compounds such as triclosan (5-chloro-2-(2,4-dichlorophenoxy)phenol) and quaternary ammonium compounds. However, other antimicrobial compounds not specifically mentioned herein are considered to be included within purview of the present invention.

Polysaccharides suitable for use in the composition according to the invention include, but are not limited to chitosan, guar, hydroxypropyl guar and starch. A “cationic polysaccharide” is a polysaccharide having positively charged sites. The cationic charge on the cationic polysaccharide may be derived from ammonium groups, bound transition metals, and other positively charged functional groups. Chitosan is believed to be the only naturally occurring cationic polysaccharide. Guar, hydroxypropyl guar, and starch are not naturally charged. However, guar, hydroxypropyl guar, and starch may be “cationized” by chemical quaternization (alkoxylation with a quaternary epoxide). The process can be performed on other types of polysaccharides besides guar, hydroxypropyl guar, and starch. Chitosan may be available from Vanson HaloSource, Inc. Cationic starch may be available from suppliers, such as AE Staley. Cationic guar may be available from suppliers, such as Hercules or Multi-Chem Corporation.

Chitosan is a polysaccharide derived from the material that forms part of the hard outer exoskeleton, especially of insects and crustaceans. Chitosan as used herein refers to copolymers having greater than 50% 2-deoxy-2-aminoglucose monomeric units with the remainder monomeric units being 2-deoxy-2-acetamidoglucose units. Chitosan can be derived from chitin by hydrolysis of some 2-deoxy-2-acetamidoglucose units to 2-deoxy-2-aminoglucose units. Due to the presence of free amino groups, chitosan is soluble in aqueous acidic solutions and is present in such media as a polycation with the protonated amino group bearing a positive charge. Reference is made to U.S. Pat. Nos. 3,533,940; 5,204,452; and 5,362,717, all expressly incorporated herein by reference. In contrast to the GB 2,107,340 application, the present invention provides a solution of chitosan without a surfactant to assist with dissolution. The sanitizing composition according to the invention, however, can include a non-ionic surfactant, a cationic surfactant or detergents to disrupt the membranes of microorganisms, such as by dissolving the lipids of the membranes, which will provide greater biocidal activity.

Guar, or clusterbean, generally refers to any polysaccharide derived from the legume (Cyamopsis tetragonoloba). Starch can be derived from many sources, including plant tubers, such as potatoes, and seed endosperms, such as wheat and corn. Starch generally refers to the polysaccharide having amylose and amylopectin molecules, both of which are polymers of α-D-glucose units.

The composition according to the invention is made from one or more of the cationic polysaccharides present in amounts of about 0.01% by weight to about 10% by weight. All percents are given as weight percents, based on the sum of the weights of all the components used to make the composition, unless otherwise indicated. The preferred range of the one or more cationic polysaccharides is about 0.1% by weight to about 2% by weight. A highly preferred range of the one or more cationic polysaccharides is about 0.5% to about 1.5%. The composition comprises at least 60% alcohol by volume. The composition comprises alcohol from about 60% by volume to about 90% by volume. The preferred range for alcohol is about 60% by volume to about 70% by volume. The composition may include one or more organic acids, or the salts thereof, to aid in the dissolution of the cationic polysaccharide in water. The amount of the one or more organic acids, or their salts, is about 0.04% by weight to about 10% by weight. A representative organic acid is lactic acid. A preferred range of lactic acid is about 0.2% by weight to about 2% by weight. Water comprises about 5% by weight to about 35% by weight. The composition may optionally contain zinc. If added, zinc comprises about 0.1% by weight to about 10% by weight. A preferred range for zinc is about 0.5% by weight to about 5% by weight. Pyrithione is another optional component in the composition. If added, pyrithione comprises 0.1% by weight to about 10% by weight. A preferred range of pyrithione is about 0.5% by weight to about 5% by weight.

One method of making the composition according to the invention includes dissolving the cationic polysaccharide in water. Depending on the nature of the cationic polysaccharide, the pH of the water may need to be adjusted with an organic acid to render the cationic polysaccharide soluble in water. For example, chitosan is cationic at a pH of less than about 8.3, meaning that chitosan will have some protonated amine groups. Above a pH of 8.3, substantially all of the amine groups present on the chitosan polymers are neutralized. In the present invention, it is preferred that chitosan be capable of dissolving in water at the stated proportions. At a pH of about 6.4, the chitosan polymer has some protonated amine groups and some neutralized amine groups, but the overall positive charge at a pH of about 6.4 is still not sufficient to keep the polysaccharide in solution. An organic acid, such as lactic acid, may be added to the water. The purpose of the organic acid is to adjust the pH of the water to a value below a pH of 6.4 in order to protonate the primary amine groups on the chitosan polymer. Below a pH of about 6.4, the number of protonated amine groups can be sufficient to render chitosan soluble in the water. Chitosan in its free base form, i.e., non-protonated, is substantially insoluble. When the pH of the water is below 6.4, the chitosan polymer has a sufficient percentage of the protonated amine groups to allow the chitosan to dissolve in the water. Above a pH of about 6.4, the chitosan polymer does not carry enough charge to keep the polymer in solution, and therefore the chitosan polymer can precipitate from the water.

Other organic acids besides lactic acid can be used. Representative organic acids may include, but are not limited to, lactic acid, pyrrolidonecarboxylic acid, citric acid, acetic acid, glutamic acid, malic acid, succinic acid, fumaric acid and glucuronic acid, or combinations. Lactic acid is preferred. The salt, chitosan lactate, is considered to be one of the least harsh on human skin as compared to other salts, such as chitosan acetate. Lactic acid usually comes as an aqueous solution. A readily available formulation of lactic acid and water for use in the present invention comprises about 88% by weight lactic acid and 12% by weight water. The percentage of water in the lactic acid is considered when adjusting the amount of water in the overall composition. Pyrrolidonecarboxylic acid is also gentle on skin.

After addition of the organic acid, an alcohol can be added to the composition. In direct contrast to the prior art, the present invention results in a solubilized form of chitosan, water and alcohol. Representative alcohols, include, but are not limited to ethanol and isopropanol, or combinations of ethanol and isopropanol. The isopropanol can be used as a denaturing agent. Alcohol is used for its sanitizing and cleansing properties. Alcohol may come in proportions of less than 100%, and may have some amount of water present. For example, an ethanol and water mixture suitable for use in the composition comes in a 95/5 (v/v) ratio. Any water present in the alcohol may be taken into account to achieve the desired amount of water in the overall composition.

Some of the optional components that may be added in the alcohol-based sanitizing composition according to the present invention include transition metals, such as zinc and copper, and/or chelating agents, such as pyrithione, to ligate with the metals and to produce bioactive complexes. Transition metal ions are known to exhibit antimicrobial activity. See, for example, U.S. Pat. No. 5,541,233, expressly incorporated herein by reference. Transition metal ions alone or in combination with a chelating agent can form complexes with chitosan, such as a zinc-chitosan chelate. It is possible that pyrithione may be omitted, since the zinc-chitosan chelate will exhibit enhanced antimicrobial activity and persistence, as compared with chitosan alone. The purpose of the pyrithione is to provide an additional chelating agent of the zinc ions. Pyrithione is in the class of thiazole compounds. Pyrithiones are sold as zinc or sodium salts, which can be obtained from the Olin Corporation under the designation OMADINE®. Cationic surfactants, non-ionic surfactants or detergents, can optionally also be included in the sanitizing composition according to the invention to disrupt the membranes of microorganisms, thereby increasing the effectiveness of the sanitizing properties of the composition. Representative surfactants that can be included in the sanitizing composition include cationic detergents (Cetyltrimethylammonium bromide; Tetradecylammonium bromide; dodecylpyrimidinium chloride); ampholytic detergents (Palmitoyllysolecithin; dodecyl-N-betaine); non-ionic surfactants [Polyoxyethylene alcohols(examples are Brj series, Lubrol W); polyoxyethylene isoalcohols(examples are Sterox AJ, Emulphogen); Polyoxyethylene p-t-octyl phenols (examples are Triton X series, Nonidet P 40); Polyoxyethylene nonylphenols (examples are Triton N series Igepal CO series); Polyoxyethylene esters of fatty acids (examples are Sterox CO series, Myrj series, Span series); Polyoxyethylene sorbitol esters (examples are Tween series, Emasol series); Ethyloxylated products such as fatty alcohol ethoxylates, fatty acid ethoxylates, fatty amide ethoxylates, monoglyceride ethoxylates, diglyceride ethoxylates and sorbitan ester ethoxylates.

The surfactants/detergents described above are not described by the general formulas put forth in the GB 2107340A patent application. The surfactants described therein are anionic and are meant to serve as counter anions of the chitosan salt and provide a anionic surfactant that is compatible with chitosan and would not lead to precipitation of chitosan.

According to the present invention, the inclusion of cationic, zwitterionic(ie. ampholytic) and non-ionic detergents/surfactants into the sanitizing composition is to provide a enhanced sanitizing effect by utilizing the detergents to disrupt microorganism membranes.

The composition of the invention can be applied on any surface to reduce the microbial population on the surface. The composition of the invention is useful in sanitizing human skin, for example. Once the alcohol has evaporated, the composition according to the invention prevents the repopulation of microorganisms on the surface for an extended period, even after the alcohol has evaporated.

### EXAMPLE 1

### Preparation of Chitosan Alcohol Gel Sanitizer

An alcohol-based hand gel containing chitosan was prepared in the following manner. One gram of chitosan (dry wt. basis) was added to 33 grams of distilled water and mixed for 10 minutes. One gram of dl-lactic acid 88% (12% water) was then added, and the solution was mixed until the chitosan dissolved. Once the chitosan was dissolved, 65 grams of a 95/5 (v/v) ethanol/water solution was slowly added to the chitosan solution while mixing.

### EXAMPLE 1A

### Preparation of Chitosan Alcohol Gel Sanitizer

An alcohol-based hand gel containing chitosan was also prepared in the following manner. One gram of chitosan (dry wt. basis) was added to 34.98 grams of distilled water and mixed for 10 minutes. One gram of dl-lactic acid 88% (12% water) was then added, and the solution was mixed until the chitosan dissolved. Once the chitosan was dissolved, 60 grams of a 95/5 (v/v) ethanol/water solution and 2.92 grams of isopropyl alcohol was slowly added to the chitosan solution while mixing. 62.91 grams of “specially denatured ethyl alcohol” (S.D.A.) formula No. 3-C could be used instead of the 60 grams of 95/5 (v/v) ethanol/water and 2.92 grams of isopropyl alcohol.

### EXAMPLE 2

### Bacterial Challenge Testing of Alcohol Gel Sanitizer Coated Glass

A comparison of the antimicrobial persistence of a dry residue of chitosan containing alcohol gel sanitizer from Example 1 to a non-chitosan containing alcohol gel sanitizer was performed as follows.

Sterile glass microscope cover slips were coated with either the chitosan alcohol gel sanitizer described in Example 1 or a commercially available chitosan-free alcohol gel sanitizer (consisting of 62% aqueous ethanol formulation). A thin uniform coat of each gel was applied with a pipette tip to one side of separate sterile glass cover slips 24×40 mm in size and allowed to air dry overnight in a sterile laminar flow hood.

Dried coated cover slips were then challenged with 50 μl of a 3.0×10⁴ CFU/ml suspension of E. coli bacteria. A sterile glass cover slip was placed on top of the 50 μl suspension and the coverslips containing the sandwiched E. coli suspensions were incubated and allowed to interact for 4 hours. After the 4 hour incubation, the coverslips were transferred to separate 50 ml conical centrifuge tubes containing 14 ml of Dulbecco's Phosphate Buffered Saline (DPBS) and vortexed for 1 minute. Vortexed solution was then diluted and plated onto Tryptic soy agar plates and incubated. Following incubation, recovered colonies were counted and recorded. Results are shown in Table 1.

TABLE 1
BACTERIAL CHALLENGE TESTING OF ALCOHOL GEL
SANITIZER COATED GLASS

| | Challenge | Recovered | | |
|---|---|---|---|---|
| | E. coli count | E. coli count | Percent | Percent |
| Coverslip Coating | CFU/ml | CFU/ml | Reduction | Increase |
| Non-chitosan alcohol | 3.0 × 10⁴ | 4.6 × 10⁴ | none | 53 |
| gel | | | | |
| Non-chitosan alcohol | 3.0 × 10⁴ | 7.2 × 10⁴ | none | 140 |
| gel | | | | |
| Chitosan alcohol gel | 3.0 × 10⁴ | 3.0 × 10³ | 90 | none |
| Chitosan alcohol gel | 3.0 × 10⁴ | 4.3 × 10³ | 86 | none |

The data in Table 1 shows that the dry coverslip coated with chitosan containing alcohol gel reduces bacterial growth after 4 hours by an average of 88%. This is in contrast to the coverslips coated with a non-chitosan containing alcohol gel which showed no reduction in bacterial growth but instead showed an increase in bacterial growth after 4 hours by an average of 97%.

This finding demonstrates the persistence property of an alcohol gel sanitizer containing chitosan.

### EXAMPLE 3

### Preparation of a Cationic guar Alcohol Gel Sanitizer

An alcohol-based hand gel containing cationic guar was prepared in the following manner. One gram of cationic guar (dry wt. basis) was added to 33 grams of distilled water and mixed for 10 minutes. One gram of dl-lactic acid 88% was then added and the solution was mixed until the cationic guar dissolved. Once the cationic guar was dissolved, 65 grams of a 95/5 (w/v) ethanol/water solution was slowly added to the cationic guar solution while mixing.

### EXAMPLE 4

### Preparation of a Cationic Starch Alcohol Gel Sanitizer

An alcohol-based hand gel containing cationic starch was prepared in the following manner. One gram of cationic starch (dry wt. basis) was added to 33 grams of distilled water and mixed for 10 minutes. One gram of dl-lactic acid 88% was then added and the solution was mixed until the cationic starch dissolved. Once the cationic starch was dissolved, 65 grams of a 95/5 (w/v) ethanol/water solution was slowly added to the cationic starch solution while mixing.

### EXAMPLE 5

### Preparation of a Chitosan Alcohol Gel Sanitizer

An alcohol-based hand gel containing chitosan was prepared in the following manner. One gram of chitosan (dry wt. basis) was added to 38 grams of distilled water and mixed for 10 minutes. One gram of dl-lactic acid 88% was then added and the solution was mixed until the chitosan dissolved. Once the chitosan was dissolved, 60 grams of isopropyl alcohol was slowly added to the chitosan solution while mixing.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

1. A sanitizing composition, comprising a soluble cationic polysaccharide and at least 60% alcohol by volume.

2. The composition of claim 1, wherein the cationic polysaccharide comprises chitosan.

3. The composition of claim 1, wherein the cationic polysaccharide comprises cationic guar.

4. The composition of claim 1, wherein the cationic polysaccharide comprises cationic starch.

5. The composition of claim 1, wherein the alcohol is at least one of ethanol, isopropanol, or the combination of ethanol and isopropanol.

6. The composition of claim 1, further comprising a transition metal.

7. The composition of claim 1, further comprising a transition metal selected from at least one of zinc or copper.

8. The composition of claim 1, further comprising a chelating agent.

9. The composition of claim 1, further comprising pyrithione.

10. The composition of claim 1, further comprising an antimicrobial compound.

11. The composition of claim 1, further comprising triclosan.

12. The composition of claim 1, further comprising a quaternary ammonium salt.

13. The composition of claim 1, wherein the composition does not include a surfactant to increase the solubility of chitosan.

14. The composition of claim 1, wherein the composition comprises a surfactant to disrupt the membrane of a microorganism.

15. A solution, comprising:
a soluble cationic polysaccharide;
at least 60% alcohol by volume;
an organic acid or a salt thereof; and
water.

16. The composition of claim 15, wherein the cationic polysaccharide is at least one of chitosan, cationic guar, or cationic starch.

17. The composition of claim 15, wherein the cationic polysaccharide is chitosan and the composition pH is below 6.4.

18. The composition of claim 15, wherein the alcohol is at least one of ethanol, isopropanol, or the combination of ethanol and isopropanol.

19. The composition of claim 15, wherein the organic acid is at least one of lactic acid, pyrrolidonecarboxylic acid, acetic acid, glutamic acid, malic acid, succinic acid, fumaric acid or glucuronic acid.

20. The composition of claim 15, further comprising a transition metal.

21. The composition of claim 15, further comprising zinc or copper.

22. The composition of claim 15, further comprising a chelating agent.

23. The composition of claim 15, further comprising pyrithione.

24. A method of sanitizing skin, comprising applying a composition to skin, wherein the composition comprises a soluble cationic polysaccharide and an alcohol.

25. The method of claim 24, wherein the composition comprises at least 60% alcohol by volume.

26. A method for making a sanitizing solution, comprising:
adding a cationic polysaccharide to water;
adding an organic acid to render the cationic polysaccharide soluble in the water; and
then adding alcohol to the water to form a sanitizing solution.

27. The method of claim 26, wherein the solution comprises at least 60% alcohol by volume.

28. A method of rendering a surface biocidal, comprising:
applying a solution of a cationic polysaccharide, alcohol, acid and water, to a surface; and
allowing the water and alcohol to evaporate to produce a biocidal film on the surface.

29. The method of claim 28, wherein the solution comprises at least 60% alcohol by volume.
